(19) United States
(12) Patent Application Publication
(10) Pub. No.: US 2004/0254560 A1
(43) Pub. Date: Dec. 16, 2004

(54) Rupture resistant blow molded freezer bag for containing blood products

(76) Inventors: Philip H. Coelho, El Dorado Hills, CA (US); Phillip Kingsley, Mather, CA (US)

Correspondence Address: BERNHARD KRETEN, ESQ & ASSOCIATES, 1331 GARDEN HIGHWAY, SUITE 300, SACRAMENTO, CA, 95833, US

(21) Appl. No.: 10/461,050

(22) Filed: Jun. 11, 2003

(51) Int. Cl. A61B 19/00; A23P 1/00

(52) U.S. Cl. 604/408; 425/383

(57) ABSTRACT

A rupture resistant medical product freezer bag and method formed by blow molding.

### FIELD OF THE INVENTION

The following invention relates generally to instumentalities and methodologies preventing bags which contain blood products from fracturing due to the extreme temperature excursions that are associated with storing the blood product in liquid nitrogen. More specifically, the instant invention is directed to a method and article of manufacture for providing a blood bag.

### BACKGROUND OF THE INVENTION

Liquid nitrogen is the preferred storage medium for many cellular blood products because its very low temperature appreciably extends the shelf life of the cellular blood product. While handling liquid nitrogen at its extreme temperatures requires a considerable degree of engineering skill, the engineering that is required to construct a bag within which the blood product is to reside has eluded even the largest and most sophisticated medical product manufacturers.

One problem which has confounded the industry in general specifically involves the areas where edges of the plastic bags are joined together. These seams are typically made using radio frequency welding. During the extreme temperature excursion associated with immersion in liquid nitrogen, these seams are susceptible to fracture. Please see the appended recent announcements by Baxter regarding the long standing problem which has evaded solution by one of the largest healthcare and medical device corporations in the world.

Applicant has already resolved this long standing problem by providing bags formed from plastic which responds to heat and pressure and retains its shape in a vacuum forming process. The plastic is formed as a shell in the vacuum forming process. Shell segments, preferably halves, are seamed together along a peripheral wall which circumscribes the outer peripherary of each shell half. A transition between a major wall of the bag and the peripheral shelf is interrupted by a radiused curve which helps distribute the forces associated with the temperature extremes common when using liquid nitrogen. This bag is very effective. People using this bag report extremely few seam failures caused by the temperature excursion in liquid nitrogen. These bags, however, are somewhat labor intensive and therefore expensive and the exterior seal remains a cause of concern for bag failure. As a consequence, their utility has been limited mainly to “exotic” applications such as stem cell preservation, where the bag cost is not the primary consideration. However, for common blood storage situations which benefit from liquid nitrogen storage, commonly used bags require a more economical method of manufacture and a reduction in the possibility of bag failure to zero.

The following prior art reflects the state of the art of which applicant is aware and is included herewith to discharge applicant's acknowledged duty to disclose relevant prior art. It is stipulated, however, that none of these references teach singly nor render obvious when considered in any conceivable combination the nexus of the instant invention as disclosed in greater detail hereinafter and as particularly claimed.

| PATENT NO. | ISSUE DATE | INVENTOR |
|---|---|---|
| 6,146,124 | Nov. 14, 2000 | Coelho et al. |

### OTHER PRIOR ART—NON PATENT LITERATURE DOCUMENTS

BAXTER, Article titled “Correct Utilization of Cryocyte Freezing Containers” (8 pages)

### SUMMARY OF THE INVENTION

The instant invention provides a bag which can withstand the rigors of low temperature excursions such as in liquid nitrogen and reduce the cost of fabrication by a factor of ten.

The technique which engenders this form of economic efficiency and reduction in cost stem from making the freezer bags in a special manner using blow molding techniques. Surprisingly, it has been discovered by applicant that the parting lines formed in a bag which are transferred from mold segments in the blow molding process (when the segments are brought into physical registry during the molding process) can withstand extreme temperature excursions common with liquid nitrogen when manufactured according to the present invention. One reason appears to be that there is very little difference between the structural integrity of the plastic at the juncture of mold segments and the constructed walls elsewhere formed within the mold cavity. In other words, the mold parting line does not demark an area of weakness because the plastic located at the site of mold segments is indistinguishable from the plastic elsewhere.

In practice, a quantum of plastic material in a semi-molten state is introduced into an open mold. Subsequently, the segments defining the mold close forming a mold cavity. Next, a gas is introduced to the center of the molten plastic such that the plastic expands to the confines imposed by the mold segments which define the cavity. Preferably, while still under gas pressure, the mold segments are modified in temperature to allow the plastic to set not only more rapidly but also to relieve stress in the formation. Once the plastic has set, the mold segments part and the bag is formed.

### OBJECTS OF THE INVENTION

It is the primary object of the present invention to provide a new and novel method for forming bags susceptible to extreme temperature excursions and the bag formed thereby.

A further object of the present invention is to provide a device as characterized above which is substantially less expensive to fabricate than in the prior art.

A further object of the present invention is to provide a device as characterized above which can withstand extreme temperature excursions in liquid nitrogen.

A further object of the present invention is to provide a device as characterized above which is extremely durable in construction and lends its self to mass production techniques.

A further object of the present invention is to provide a bag which is to be exposed to temperature extremes without suffering fracture at sites of historical weakness.

Viewed from a first vantage point it is an object of the present invention to provide a rupture resistant medical freezer bag formed for storing blood product at a depressed temperature such that the blood product changes phase from a liquid to a solid and then back to a liquid, the bag comprising, in combination: an enclosure for the blood product having a pair of first and second parallel, spaced side walls interconnected by a peripheral edge wall circumscribing a periphery of each said side wall, said edge wall including a discernable parting line caused by the bag having been formed in an openable mold.

Viewed from a second vantage point is an object of the present invention to provide a rupture resistant medical freezer bag for storing a cellular blood product at a depressed temperature such that the blood product changes phase from a liquid to a solid, the bag formed by: introducing a plastic into an area flanked by mold segments, closing the mold segments such that when the mold segments are brought together the plastic is surrounded by the segments whose interior conforms to the shape of the bag to be formed, injecting a gas into the plastic such that the plastic expands up to the interior confines of the mold causing the plastic to conform to the mold interior, setting the plastic to hold the shape of the mold, and removing the bag thus formed from the mold.

These and other objects will be made manifest when considering the following detailed specification when taken in conjunction with the drawing figures.

### DESCRIPTION OF THE DRAWINGS FIGURES

FIG. 1 is a perspective view of the blow molded bag according to the present invention.

FIG. 1A is a sectional detail of one aspect shown in FIG. 1.

FIG. 2A is a side view showing an attachment feature of one portal.

FIG. 2B is an end view of that which is shown in FIG. 2A

FIG. 3A is perspective of the mold used to form the bag of FIG. 1.

FIG. 3B is a sectional view of the mold schematically depicting a first stage in the molding process.

FIG. 3C shows a second stage with respect to FIG. 3B.

FIG. 3D depicts a third stage in the blow mold process.

FIG. 3E shows the resulting bag in perspective as formed prior to trimming.

FIG. 4A shows a ferrule in section.

FIG. 4B shows the ferrule being inserted within the bag using a tool.

FIG. 4C shows the ferrule being located in the bag.

FIG. 4D shows the ferrule being oriented for sealing engagement within the bag.

FIG. 4E shows the ferrule being sealed in place.

FIG. 4F is a side view of that which is shown in FIG. 4E.

FIG. 4G shows a second seal being formed over the ferrule.

FIG. 4H shows the second seal after forming.

FIG. 4I is a side view of that which is shown in FIG. 4H.

FIG. 5 is flow chart of the methodology according to the present invention.

### BRIEF DESCRIPTION OF THE PREFERRED EMBODIMENT

Considering the drawings, when like numerals denote like parts, numeral 10 is directed to the bag according to the present invention and numeral 100 is directed to the mold.

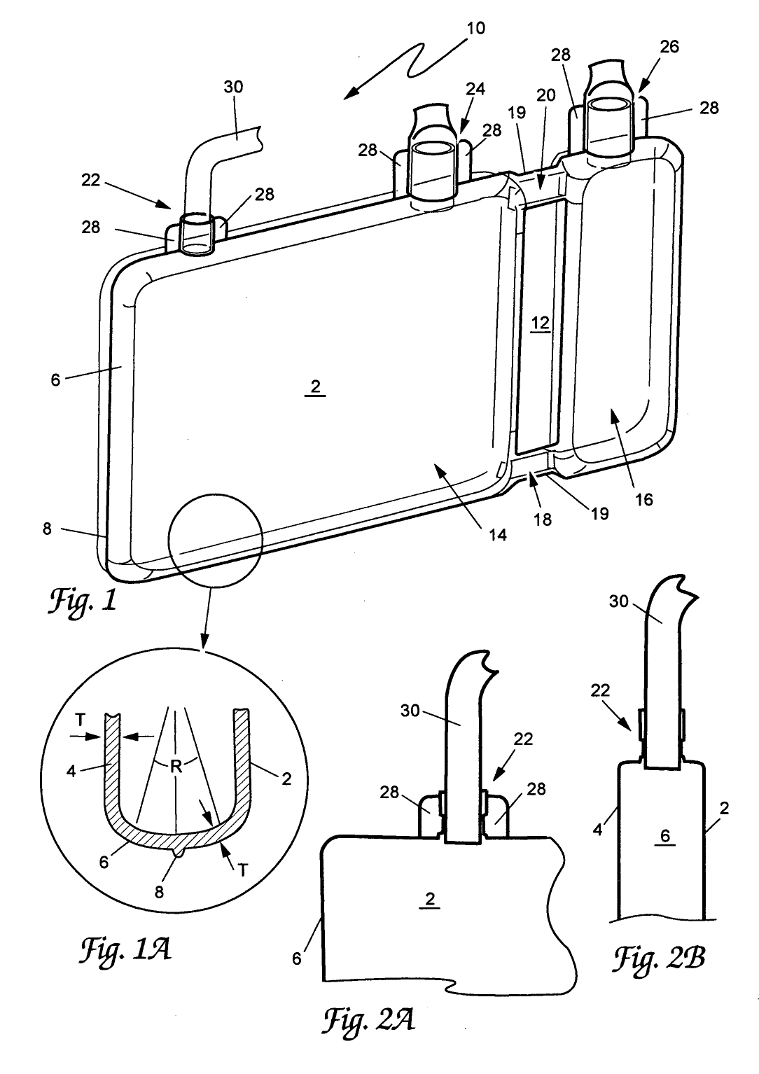

Considering the bag 10, it is formed from a blow molding process resulting in a first planar side wall 2, a second planar side wall 4 and a peripheral side wall 6 which circumscribes the first and second side walls 2, 4. The peripheral side wall 6 is radiused such that a constant radius of curvature R (FIG. 1A) is provided such that the wall has constant thickness T along every aspect of the bag and specifically the side walls so that there are no zones of structural weakness. Blow molding tends to assure the side wall (historically a weak area) is as robust as the other bag walls. FIG. 1A also makes clear the existance of a parting line 8 which will be described in greater detail in conjunction with the formation using the blow mold.

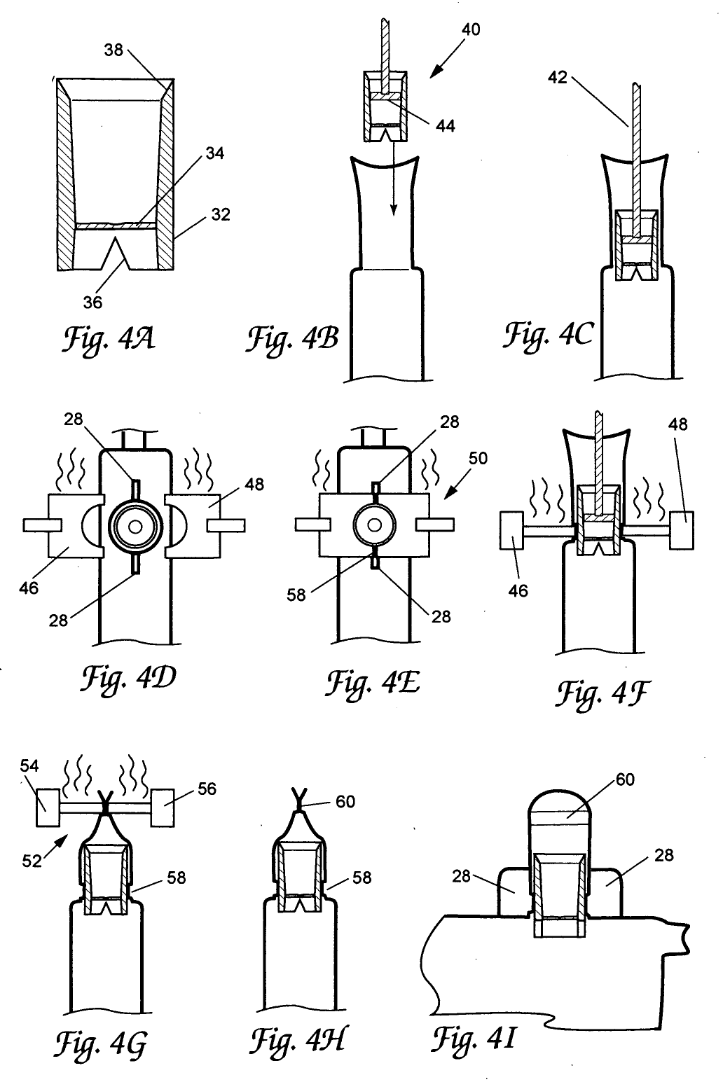

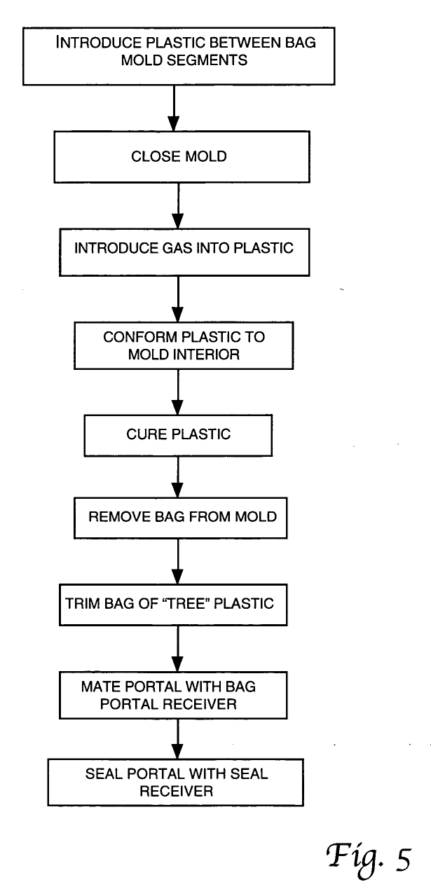

An illustrative bag includes a partition 12 which provides a demarcation between a first storage area 14 and a second storage area 16. The two storage areas 14, 16 are interconnected by a first passage way 18 and a second passage way 20. These two passage ways respectively straddle upper and lower portions of the partition 12 providing fluid communication between the first storage area and the second storage area. Each storage area is provided with its own portal. Specifically, the first storage area 14 includes a first portal 24. Similarly, second storage area 16 includes second portal 26. These portals are used to remove product from within the interior of the bag 10. A third portal 22 is used to introduce product into the bag. In practice, the third portal receives the product and the product is distributed between the first storage area and second storage area by means of the first and second passage ways. Subsequently, if the storage areas do not require simultaneous deployment, the first and second passage ways 18, 20 are heat sealed providing a barrier between the storage areas so that either storage area can be utilized via its own portal. Both the first and second portals 24, 26 are reinforced with a ferrule 32 (FIG. 4A) which is provided with a dam 34 contained within the ferrule and which must be punctured in order to access the contents of the bag 10. The ferrule is substantially cylindrical in shape and includes a V shape notch 36 inverted on a bottom wall thereof and a chamfer 38 on a top wall. The V shape notch, when inserted into the bag 10, lies in registry with an interior of the peripheral side wall so that the apex of the inverted V is in substantially the same plane as the interior of the radiused side wall, allowing complete extraction of all of the contents within the bag. The third portal 22 is provided with an inlet tube 30 to facilitate in the introduction of the liquid to within the interior of the bag.

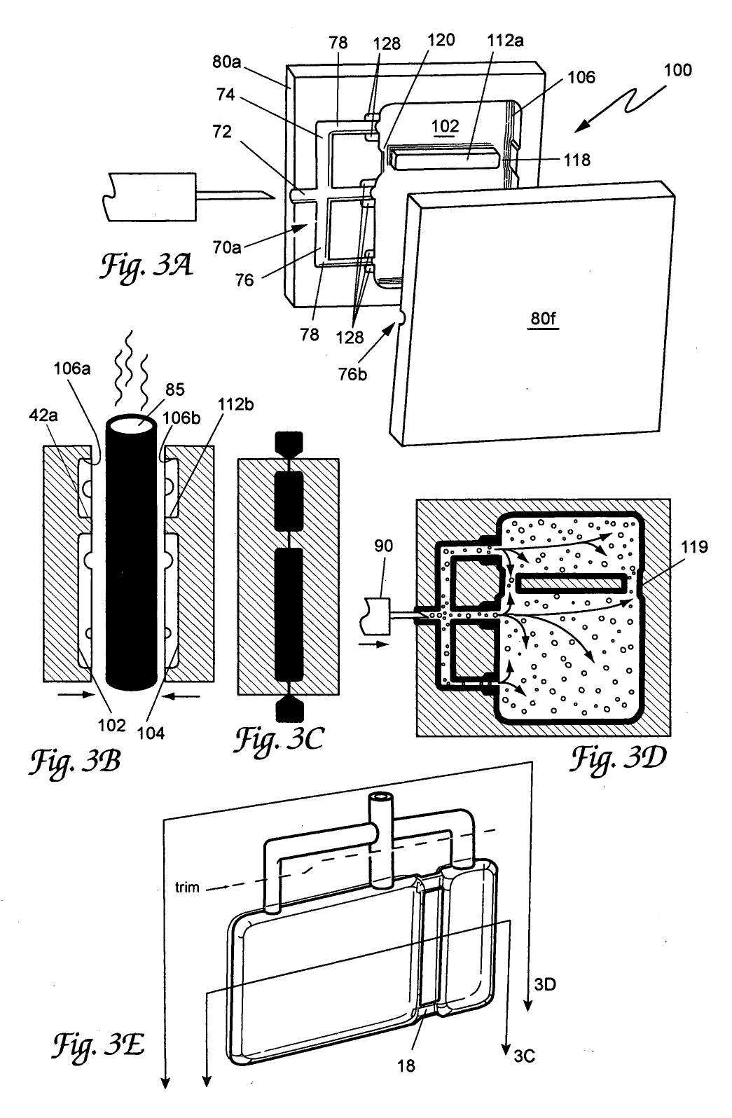

FIGS. 3A through D show the mold which forms the bag of FIG. 1. The mold 100 is formed from a plurality of segments, in the illustrative example two segments 80A and 80B. First segment 80A as shown in FIG. 3A has a mirror image in second segment 80B whose back is shown so that all sides of the mold are perspicuous. Thus, each segment includes a first planar wall 102 and a radiused side wall 106. The dimension of the side wall 106 is half of the radiused peripheral side wall 6 which ultimately forms the bag. Also shown is a raised area 112 which forms the partition 12. The raised area 112 allows the formation of the first and second passage ways 18 and 20 by virtue of the gaps 118 and 120 at longitudinal extremities of the partition 112. Note that gaps 118 and 120 which lead to the radiused peripheral side wall 6 of the bag includes a step 119 which provides a complemental neck 19 in the peripheral side wall of the bag at the first and second passage ways 18 and 20. By having a necked down portion, the passage ways are more easily sealed. As mentioned earlier, this allows sequestration of the first storage area 14 from the second storage area 16.

A manifold 70 is also formed into the mold 100. As shown, the manifold is formed from two halves, a first half 70A and a second half 70B which is the mirror image of the one detailed in FIG. 3A. The manifold defines a tree that allows forming of the portal to be described infra. The manifold 70 includes a main conduit 72 whose axial extension leads to the first portal 24 of the bag. In addition, the manifold includes a first lateral branch 74 and a second lateral branch 76 emanating transversely from the main conduit 72. The lateral branches 74 and 76 include elbows 78 to redirect the branches into parallel relationship with the conduit 72 so that the branch 76 addresses the third portal 22 and the branch 74 addresses the second portal 26.

FIG. 3B shows the mold in an open condition to receive a slug 85 of the plastic material that is to form the bag. Once the slug 85 has been placed in the confines of the mold 100, the mold is closed (FIG. 3C) and the exterior contour of the bag is formed. With reference to FIG. 3D, the manifold 70 receives a parison 90 which injects a gas through the manifold and into the interior of the mold. This forces the plastic slug 85 to become hollow with the plastic conforming to the confines of the interior mold thereby producing a bag having an exterior skin complemental to the interior surface of the mold. As shown in FIG. 3D, the mold is preferably thermally conductive and allows heat transferred ΔT to occur to control the temperature of the mold and therefore that of the bag as it has been formed. This temperature control is critical for a multiplicity of reasons. For example, precise temperature control of the mold optimizes cycle time in product formation. That is, by carefully controlling the temperature profile of the mold, product throughput can be optimized. In addition, however, a corollary to the mold temperature control also involves the potential for stress relieving the formed article by controlling the temperature excursion the molded bag experiences prior to removal.

In any event, the finished article of manufacture as shown in FIG. 1 is achieved after the FIG. 3E article has been trimmed of the manifold as shown by the trim line in that figure. The final steps are shown in FIG. 4 which delineate the manner in which the ferrule 32 is to be inserted within any of the manifold's upstanding portions which have survived the trimmings shown in FIG. 3E.

As mentioned, each ferrule includes a chamfer 38 at one end. This allows location of the insertion tool 40 by self centering via the chamfer. The insertion tool 40 includes a stem 42 which communicates with a retaining disk 44. Retaining disk frictionally holds the ferrule 32 and places the ferrule into the conduits and branches which are to form the portals. Once the ferrule has been inserted such that the notch 36 registers so that it's apex is coplanar with the inner peripheral wall, a first heating anvil 50 (formed from a first U shaped horn 46 and a second U shaped horn 48) is poised (FIG. 4D) to bond the ferrule into the untrimmed material left from the manifold that is to form the portal. By “heating” is meant thermal, sonic or RF excitation. Notice that the mold had been provided with tab forming recesses 128 adjacent to juncture of a manifold to the bag. These tab forming recesses 128 result in tabs 28 being formed on the peripheral outer surface and projecting up coplanar with the parting line. As the horn 50 prepares to close as shown in FIG. 4E the tab 28 is interposed between the horn elements, preventing arcing of the horns. FIG. 4F is a side view thereof. Once the ferrule has been united to the bag, the insertion tool 40 can be removed. Next, the opening which exists after the removal of the insertion tool 40 is sealed by means of a second heating anvil 52 having a first planar element 54 and a second planar element 56. These anvil elements unite to form a second bond 60 in conjunction with the first bond 58 providing a sealed access area assuring aesepsis for the bag.

Moreover, having thus described the invention, it should be apparent that numerous structural modifications and adaptations may be resorted to without departing from the scope and fair meaning of the instant invention as set forth here and above and as defined by the claims here and below.

1. A rupture resistant medical freezer bag formed for storing blood product at a depressed temperature such that the blood product changes phase from a liquid to a solid and then back to a liquid, the bag comprising, in combination:
an enclosure for the blood product having a pair of first and second parallel, spaced side walls interconnected by a peripheral edge wall circumscribing a periphery of each said side wall, said edge wall including a discernable parting line caused by the bag having been formed in an openable mold.

2. A rupture resistant medical freezer bag for storing a blood product at a depressed temperature such that the blood product changes phase from a liquid to a solid, the bag formed by:
introducing a plastic into an area flanked by mold segments,
closing the mold segments such that when the mold segments are brought together the plastic is surrounded by the segments whose interior conforms to the shape of the bag to be formed,
injecting a gas into the plastic such that the plastic expands up to the interior confines of the mold causing the plastic to conform to the mold interior,
setting the plastic to hold the shape of the mold, and
removing the bag thus formed from the mold.
